(19) United States
(12) Patent Application Publication
(10) Pub. No.: US 2010/0209486 A1
(43) Pub. Date: Aug. 19, 2010

(54) Method of enhancing CoenzymeQ10 levels in mammals

(76) Inventors: Ganga Raju Gokaraju, Vijayawada (IN); Rama Raju Gokaraju, Vijayawada (IN); Ranga Raju Gokaraju, Vijayawada (IN); Golakoti Trimurtulu, Vijayawada (IN); Kiran Bhupathiraju, Vijayawada (IN)

Correspondence Address: KRAMER & AMADO, P.C., 1725 DUKE STREET, SUITE 240, ALEXANDRIA, VA, 22314, US

(21) Appl. No.: 12/388,281

(22) Filed: Feb. 18, 2009

(51) Int. Cl. A61K 9/127; A61K 31/7076; A61K 9/00; A61K 8/02

(52) U.S. Cl. 424/450; 424/94.1; 424/400; 424/48

(57) ABSTRACT

The invention relates to a method of enriching the CoEnzyme Q₁₀ levels in mammals through supplementing CoQ₉ or the compositions containing CoQ₉. The present invention further relates a therapeutic method for obtaining potent antioxidant, cardioprotective, immunomodulating anticancer effects similar to those obtained with CoQ₁₀ supplementation, by enhancing the CoQ₁₀ levels by supplementing the mammal with CoQ₉ or nutraceutical compositions or dietary supplements or pharmaceutical formulations comprising CoQ₉.

### FIELD OF THE INVENTION

The invention relates to method of enriching the CoenzymeQ₁₀ levels in mammals through supplementing CoQ₉ or the compositions containing CoQ₉. The present invention further relates to a therapeutic method for obtaining potent antioxidant, cardioprotective, immunomodulating anticancer effects similar to those obtained with CoQ₁₀ supplementation, by enhancing the CoQ₁₀ levels by supplementing the mammal with CoQ₉ or nutraceutical compositions, dietary supplement compositions and pharmaceutical formulations comprising CoQ₉.

### BACKGROUND OF THE INVENTION

Coenzyme Q₁₀ (CoQ₁₀), an endogenously synthesized pro-vitamin present in the mitochondrial electron transport chain, is a natural substance, belong to a family of 2,3-dimethoxy-5-methyl-6-polyprenyl-1,4-benzoquinone compounds, widely know as ubiquinone for its ubiquitous occurrence in animal and plant tissues. It has been found to be cardio-protective and used as adjunct therapy for ischemic heart disease (Kitamura, N., et. al., In: Biochemical and Clinical Aspects of Coenzyme Q. Folkers, K., Yamamura, Y., Eds.; Elsevier, Amsterdam, Netherlands vol 4, pp 243-256). Mitochondrial respiratory chain contains several coenzymes including Coenzymes Q₁, Q₂, Q₄, Q₆, Q₇, Q₈, Q₉ and Q₁₀. Coenzymes Q₆, Q₇ and Q₈ exist in yeast and bacteria, whereas, CoQ₁₀ is prevalent in humans. CoQ₉, on the other hand, is found in rats and mice. The CoQ₉ differs from CoQ₁₀ with respect to the number of isoprenoid units in the tail. CoQ₉ has nine isoprene units in the side chain in contrast to the presence of 10 units in CoQ₁₀.

The CoQ₁₀ is an essential cofactor for the proper functioning of uncoupling proteins. In addition to its important role as a redox component for both mitochondria and lipid membrane, CoQ₁₀ in the reduced form (ubiquinol) functions as an antioxidant, which protects biological membranes and serum LDL from lipid peroxidation. CoQ₁₀ also serves as a powerful antioxidant in other organelle membranes that contain CoQ (Overvad, K., et. al., Eur. J. Clin. Nutr. 1999, 53, 764-770).

Most of the CoQ₁₀ are found in mammalian hearts including human myocardium (Folkers, K., et al., Proc. Natl. Acad. Sci. USA 1985, 82(3), 901-904). CoQ₁₀ is not an essential nutrient because it is synthesized in the body. The CoQ₁₀ level in the body decreases with age and under several pathophysiological conditions. Several food products including meat, fish, peanuts and broccoli are the rich source of CoQ₁₀. Although the dietary intake of CoQ₁₀ is about 2-5 mg per day, it is inadequate for the body under pathophysiologic conditions and in old age.

The levels of endogenous CoQ₁₀ in the heart decreases during ischemic heart disease including heart failure and this has prompted clinical trials on CoQ₁₀ in heart patients (Otani, H., et. Al., Circ. Res. 1984, 55, 168-175). Randomized, double-blind placebo-controlled trials on oral administration of CoQ₁₀ have confirmed the effectiveness of CoQ₁₀ in improving anginal episodes, arrthythmias, and left ventricular function in patients with acute myocardial infarction (Singh, R. B., et. al., Cardiovasc. Drug. Ther. 1998, 12, 347-353).

Studies involving breast cancer patients showed that CoQ₁₀ concentrations in tumor tissues significantly depleted as compared to the surrounding normal tissues. Administration of coenzyme Q₁₀ by dietary supplementation found to induce the protection against tumor growth. The high risk breast cancer patients supplemented with 90 to 390 mg daily doses of CoQ₁₀ obtained partial to complete regression (Lockwood K, et. al. Mol Aspects Med. 15 (Suppl): s231-40, 1994). It also prevents cardiotoxicity of some of the anticancer drugs. CoQ₁₀ is an immunomodulating agent and is essential for the optimal function of the immune system (Folkers K.,; Drugs Exp Clin Res. 11(8):539-45, 1985).

A recent study showed that supplementation of CoQ₁₀ could reduce myocardial ischemic reperfusion injury in pigs on cardiopulmonary bypass. Additionally, recent studies also indicate a novel role of exogenous CoQ₁₀ in the induction and transcription of genes involved in cell signaling, metabolism and transport. Another recent study has indicated CoQ₁₀ as a modulator of transition pore suggesting its role in apoptosis. In most countries CoQ₁₀ is widely used as a nutritional supplement. In countries like Japan, it is a drug prescribed for those having suffered from heart disease. However, in USA it is a dietary supplement available from health food store or mail order business.

CoQ₉ is a lower homolog having nine isoprene units compared to ten present in CoQ₁₀. A recent study has indicated that reduced CoQ₉ could act as a potential antioxidant regardless of its cellular concentration (Ernster, L., et. al., Clin. Investig. 1993, 71, S60-S65). Reduced CoQ₉ together with α-tocopherol, were found to act as potential antioxidant in guinea pig hepatocytes when incubated with AAPH, while reduced CoQ₁₀ mainly exhibited its antioxidant activity in cells containing CoQ₁₀ as the predominant CoQ homolog. Another related study has demonstrated significant decrease of CoQ₉ in heart mitochondria of diabetic rats suggesting reduced CoQ₉ could be responsible for the increased susceptibility of diabetic heart to oxidative damage. Yet another study indicated that myocardial reperfusion decreased the mitochondrial content of ubiquinone and stimulated CoQ₉ biosynthesis in young rats but not in aged rats. The synthesis of CoQ₉ was found to be increased in the liver in hyperthyroidism. A recent study indicated that Coenzyme Q₉ could regulate the aging process in Caenorhabditis elegans mitochondria. Similar to CoQ₁₀, CoQ₉ also participates in the mitochondrial electron transport inside of the cell, and in rodents where CoQ₉ is the predominant Coenzyme Q, it serves as an essential component for the ATP synthesis.

WO07017168A1 describes a process for the preparation of ubihydroquinones and ubiquinones by condensation of a prenol or isoprenol with a hydroquinone or derivative thereof in the presence of 0.005-1.0 mol % of a catalyst which is a Broensted-acid, a Lewis-acid from the group consisting of a derivative of Bi or In or an element of group 3 of the periodic table of the elements, a heteropolyacid, an NH— or a CH-acidic compound, and optionally oxidizing the ubihydroquinone obtained.

WO07014392A2 comprises benzoquinone compositions of enhanced solubility and bioavailability that contain at least one benzoquinone with at least one solubility-enhancing polymer. In one embodiment, the benzoquinone is coenzyme Q10.

WO07004091A2 relates to novel intermediates for the preparation of coenzymes, processes for the preparation of the intermediates and an improved process for the preparation of Coenzymes, more particularly relating to regio and stereo controlled process for Coenzyme Q₉ and Coenzyme Q₁₀.

US20060010519A1 describe a plant which expresses a large amount of ubiquinone-10 and a method for producing ubiquinone-10 using the plant are provided. A dietary supplement, a food and a food additive which contains ubiquinone-10 produced by the plant or the method are provided.

JP2005041870A2 describes an external preparation having skin keratin-softening action, skin-whitening action, moisturizing action or wrinkle-removing action, comprising a ubiquinone and the deep-sea water. The examples of ubiquinones, coenzyme Q6, coenzyme Q7, coenzyme Q8, coenzyme Q9 or coenzyme Q10 is used.

US20040033553A1 describes a method to assay coenzyme Q10 in blood plasma or blood serum, wherein Q₁₀ in the plasma sample is oxidized by treating the sample with an oxidizing agent having a redox potential higher than the redox potential of CoQ₁₀, such as, for example, para-benzoquinone. Following oxidation of the CoQ₁₀, the CoQ₁₀ in the plasma sample is extracted with an alcohol, such as, for example, 1-propanol. The alcohol extract is analyzed using direct injection into the HPLC apparatus.

US20050008630A1 describes a method of stabilizing reduced coenzyme Q10, which is useful as functional nutritive foods, specific health foods and the like. Furthermore, the present invention provides a method for efficiently obtaining reduced coenzyme Q₁₀ of high quality and by a method suitable for a commercial production.

US20050137410A1 describes a practical, cost-effective synthesis of CoQ10, wherein the invention provides a convergent method for the synthesis of ubiquinones and ubiquinone analogues. Also provided are precursors of ubiquinones and their analogues that are useful in the methods of the invention.

EP0882450B1 describe a cholesterol-lowering composition comprising coenzyme Q [From equivalent EP0882450A2] provide an antihypercholesterolemic or antihyperlipidemic agent, hence a therapeutic and prophylactic drug for arteriosclerosis, which is safer and more potent in cholesterol-lowering action than the hitherto-available drugs, comprising a coenzyme Q or a reduced coenzyme Q, wherein the polyprenyl side chain of the ubiquinone contains 6 to 11 isoprenyl units.

EP1068805A1 describe a method for inhibiting blood coagulation in humans and warm blooded animals comprising administration by the oral route of cereal germ oil, preferably corn oil, wherein germ oil used for administration has been enriched in ubiquinone 9/10.

JP02249492A2 describe a process for the production of ubiquinone 9, to industrially and advantageously obtain the subject compound useful as a remedy for cerebrovascular disorder, cardiac insufficiency, hypertension or diabetes, side effect preventive agent of an anticancer agent adriamycin, etc., by culturing a microorganism belonging to the genus Mucor.

JP01117793A2 describe a process for the production of ubiquinone, which is used as a remedy for cerebrovascular disorders, cardiac failure, hypertension or the like in high efficiency by culturing a strain in Mortierella in an enriched medium.

JP61027914A2 provide a cosmetic free from undesirable side effect such as hormonic effect, and having excellent hair-tonic effect and acne-remedying effect, by compounding a specific ubiquinone with oxendlone (16μ-ethyl-17μ-hydroxy-4-estren-3-one). The ubiquinone compound is selected from ubiquinone 7, ubiquinone 8, ubiquinone 9 or ubiquinone 10.

JP59013719A2 describes an effective remedy to the prevention and remedy of male pattern baldness, etc. without causing side effects such as exhaustion of sexual energy, etc. even by the continuous long-term administration, free from hormone-like action, and effective at a low dose, by using a ubiquinone as an active component. The ubiquinone of formula (n is 7W10) is used as an active component. The ubiquinone is ubiquinone-7 (n=7), ubiquinone-8 (n=8), ubiquinone-9 (n=9) or ubiquinone-10 (n=10).

JP57202294A2 describes a process for producing coenzyme Q9, by cultivating a bacterium such as Rhodopseudomonas capsulate (FERM-P 879) belonging to the genus Rhodopseudomonas, capable of producing coenzyme Q9, when inoculated into a nutritive medium under aerobic conditions, and coenzyme Q9 is collected from the culture mold.

JP55000028A2 and JP54138191A2 also describe similar processes for producing coenzyme Q9, by cultivating a bacteriums Pseudomonas genus and Streptomyces sapporonensis respectively followed by purification techniques.

JP53072895A2 describes a process for producing coenzyme Q9 (which is coenzyme widely distributing in microorganisms, plants and animals) having an important role of electron transfer in vivo, useful as pharmaceuticals, by culture of plant cells.

U.S. Pat. No. 4,031,205 describes a method for treating nervous bladder comprising administering to a human suffering from nervous bladder a therapeutically effective amount of Ubiquinone, wherein the polyprenyl side chain of the ubiquinone contains 0 to 10 isoprenyl units whereby nervous bladder can be treated without side-effects.

None of the prior art provides a method for enhancing the CoQ₁₀ levels in a mammal through the supplementations of an ingredient other than CoQ₁₀ itself.

There exists a need for a method to enhance the concentration of CoQ₁₀ levels in mammals. It is therefore an objective of the present invention to provide a practical method, to enhance the levels of CoQ₁₀ in a mammal through the supplementation of relatively cost effective ingredients or the compositions containing such ingredients

### SUMMARY OF THE INVENTION

The present invention discloses a method for enriching the CoenzymeQ₁₀ levels in mammalian body through supplementing CoQ₉ or the compositions containing CoQ₉.

The major objective of the present invention is to provide a therapeutic method for obtaining potent antioxidant, cardioprotective, immunomodulating anticancer effects similar to those obtained with CoQ₁₀ supplementation, by enhancing the CoQ₁₀ levels through supplementing the mammal with CoQ₉ or nutraceutical compositions, dietary supplements, pharmaceutical formulations and cosmetic preparations comprising CoQ₉.

Another major objective of the present invention is to find economically cheaper alternative to CoQ₁₀.

### DESCRIPTION OF THE DRAWINGS

FIG. 1. The incidence of reperfusion-induced ventricular fibrillation (VF). Guinea pigs were orally treated with a daily dose of 5 mg/kg of CoQ₁₀ or CoQ₉ or vehicle control for 4 weeks, and then hearts were excised, and isolated for perfusion via Langendorff mode and subjected to 30 min of global ischemia followed by 120 min of reperfusion. N=12 in each group, *p<0.05 compared to the drug-free control (C) group.

FIG. 2/A Effects of CoQ₁₀ and CoQ₉ on infarct size in isolated guinea pig hearts subjected to 30 min of ischemia followed by 120 min of reperfusion. The bars represent mean±SD of infarct size for control, CoQ₁₀ and CoQ₉ supplemented groups. *p<0.05 compared to the untreated age-matched ischemic/reperfused drug-free control (C) value. N=12 in each group.

FIG. 2/B. Effects of CoQ₁₀ and CoQ₉ on cardiomyocyte apoptosis in isolated guinea pig hearts subjected to 30 min of ischemia followed by 120 min of reperfusion. The bars represent mean±SD of cardiomyocyte apoptosis for control, CoQ₁₀ and CoQ₉ supplemented groups. *p<0.05 compared to the untreated age-matched ischemic/reperfused drug-free control (C) value.

FIG. 3. HPLC chromatograms of a) Blank (mobile phase) b) CoQ₉ standard solution c) CoQ₁₀ standard solution and d) CoQ₉ heart sample. The standards and sample solution were analyzed using an Agilent 1100 HPLC. The mobile phase was methanol-2-propanol-formic acid (45:55:0.05, v/v/v) containing methylamine at the concentration of 5 mmol/L. At a flow rate of 0.2 ml/min, 5 μl injections of the samples were done using the autosampler. The CoQ₉ peak eluted at 2.39 minutes and the CoQ₁₀ eluted at 2.86 minutes. However, the CoQ₉ heart sample peak eluted at 2.89 minutes, which is the same as CoQ₁₀, thus indicating the presence of CoQ₁₀ rather than CoQ₉ in the sample. Bio-conversion of Q₉ into Q₁₀ is suggested and this is further verified using mass spectrometric analysis.

FIG. 4. Mass Spectrometry (MS). (a) CoQ₁₀ standard, b) CoQ₉ standard, c) CoQ₁₀ heart sample) Methylamine was used in the mobile phase to obtain the methyl ammonium adduct molecules of CoQ₉ and CoQ₁₀. The sensitivity of the adduct ions [M+CH₃NH₃]⁺ was much higher than that of the protonated ions [M+H]⁺ [7]. The MS spectra of both [M+CH₃NH₃]⁻ at m/z 826.5 for CoQ₉ and m/z 894.6 for CoQ₁₀ were observed. However, the CoQ₉ heart sample indicated a mass peak at m/z 894.6, which matches the peak for CoQ₁₀ and not CoQ9. Therefore, there was evidence of CoQ₁₀ in the CoQ₉ heart sample from the mass spec. data. This confirmed the hypothesis of bio-conversion of CoQ₉ to CoQ₁₀.

### DETAILED DESCRIPTION OF THE INVENTION

CoQ₁₀ is an essential component of the mitochondrial electron transport chain involved in both photosynthetic and respiratory processes. It acts as the redox link between flavoproteins and cytochromes that are essential for ATP synthesis. It also functions as an antioxidant in cell membranes and lipoproteins (Ernster, L., et al., Biochim. Biophys. Acta, 1995, 1271, 195-207) and exhibits potent clinical effect in human congestive heart failure, hypertension and cancer, in addition to wide array of other medicinal application.

Under the normal condition, body may not require any exogenous CoQ₁₀ since it is produced by de nova biosynthesis. However, in certain pathophysiologic conditions such as hypertension, cardiomyopathy, angina, heart failure, muscular dystrophy and cancer [Simonsen, R., et. al., In Biochemical and Clinical Aspects of Coenzyme Q Folkers, K., Littarru, G. P., Yamagami, T., Eds] Elsevier, Amsterdam, Netherlands, vol 6, pp 363-373 and Beyer R E, et. al., In Pathology and Cardiovascular Injurer (Stone, H. L., Weglicki. W. B., Eds), Martinus Njhoff, Boston, Mass. pp 489-511], de novo production of CoQ₁₀ is reduced and hence, tissues require exogenous supply of CoQ₁₀. The cellular CoQ₁₀ deficiency is greatly enhanced with the advancement of age. Most importantly, heart requires additional CoQ₁₀ for maintaining optimum ATP levels under pathophysiologic conditions such as ischemic heart diseases including heart failure. Correction of deficiency requires supplementation of CoQ10 at concentrations, higher than those available in the regular diet.

Although CoQ₉ is also present in the human body, CoQ₁₀ remains the only CoQ supplement that is commercially available. The CoQ₁₀ commercial supplies have now been available and widely being used as a dietary ingredient in many countries around the world. The CoQ₁₀ currently being marketed around the world is produced solely from fermentation route. Its production is acutely limited due to the monopoly. Even though several chemical processes are available for the CoQ₁₀ production, all of them are economically unviable. Because of the source limitation, there is a big fluctuation in the market price, i.e. US $3000/kg in 2005 to $800/kg in 2007, depending upon the supply and demand. Alternative products or methods or sources are greatly needed to augment the growing demand and to provide greater access to this beneficial anti-oxidant to wider cross sections of the population.

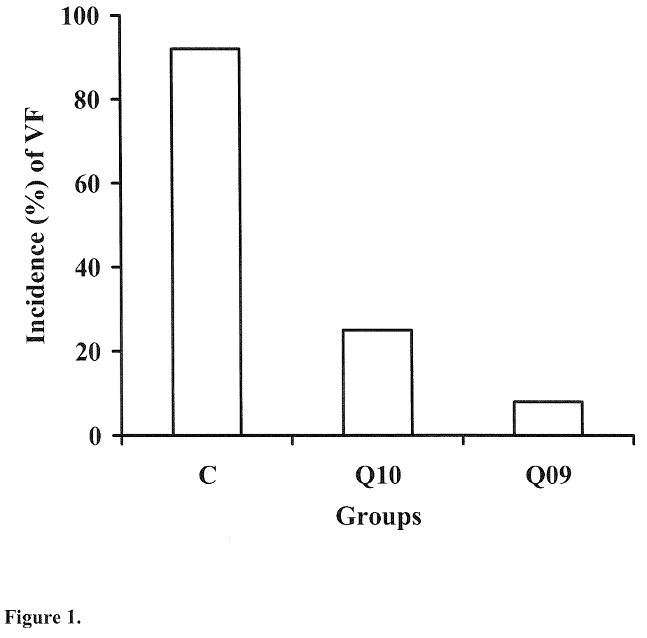

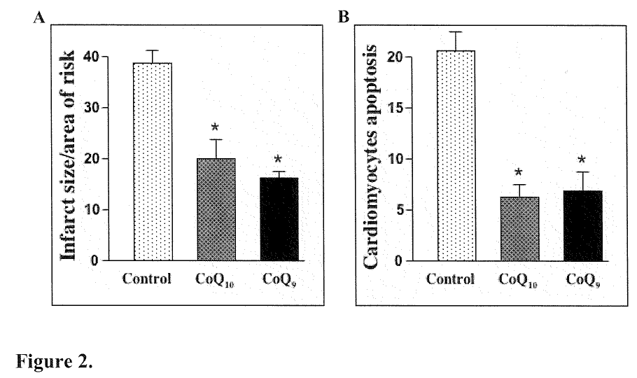

Though the chemical production of CoQ₁₀ economically unviable, the production of its lower CoQ homolog, i.e., CoQ₉ through chemical technology is cost effective. This is possible as the C45 side chain can be derived and adopted directly from a natural product called solanesol. Solanesol can be isolated from waste tobacco raw material. Whether CoQ₉ can also perform the same task for the heart and offer similar health benefits as CoQ₁₀, especially if CoQ₉ supplementation can reduce myocardial ischemia reperfusion, is not known. It is also not known whether exogenous CoQ₉ could be equally cardioprotective as CoQ₁₀ in the animals where CoQ₉ is totally absent or less predominant. The inventors performed a series of ex viva and in viva studies and compared the effects of CoQ₉ Vs. CoQ₁₀ in the ischemic myocardium, and found surprisingly that CoQ₉ could protect the ischemic heart to the same extent as CoQ₁₀ (FIGS. 1, 2A and 2B). The inventors also found most surprisingly that when a mammal is supplemented with CoQ₉, it is bio-converted into CoQ₁₀ and lead to enhancement of CoQ₁₀ concentration over and above the un-supplemented mammal. This unexpected result is likely that CoQ₉ could fill up the gap for CoQ₁₀ after being converted into CoQ10 as the bioavailability of CoQ₁₀ is very poor.

Experimental studies were designed to determine if CoQ₉ could protect guinea pig hearts from ischemia reperfusion injury. Myocardial ischemia reperfusion injury model is the most widely accepted experimental method for assessment of parameters related to cardio-protection. Guinea pigs were randomly divided into three groups: Group II and Group III were supplemented with 5 mg/kg bodyweight of CoQ₉ and CoQ₁₀, respectively, for 4 weeks while Group I served as control (C). After 4 weeks, the guinea pigs were sacrificed and the isolated hearts were perfused via working mode. The isolated hearts were subjected to ischemia for 30 min followed by 2 hours of reperfusion. Cardioprotection was assessed by evaluating left ventricular function, ventricular arrhythmias, myocardial infarct size and cardiomyocyte apoptosis. Samples of hearts were examined for the presence of Coenzyme Q. The results demonstrated that both CoQ₉ and CoQ₁₀ were equally cardioprotective as evidenced by their abilities to improve left ventricular performance (Table 1 and FIG. 1), and to reduce myocardial infarct size (FIG. 2A) and cardiomyocyte apoptosis (FIG. 2B). High performance liquid chromatographic (HPLC) analysis revealed surprisingly that a substantial portion of CoQ₉ had been bio-converted into CoQ₁₀. The results indicate that CoQ₉ by itself, or after being converted into CoQ₁₀, provides cardioprotection in myocardial ischemic reperfusion injury.

Several unexpected salient features are apparent from the present investigation. First, CoQ₉ and CoQ₁₀ provided similar magnitude of cardioprotection as evidenced from the comparable degree of the post-ischemic ventricular recovery, reduction of myocardial infarct size (FIG. 2A) and cardiomyocyte apoptosis (FIG. 2B). Both CoQ₉ and CoQ₁₀ supplementation reduced the incidence of ventricular fibrillation (FIG. 1). LC-GC results revealed complete bioconversion of CoQ₉ into CoQ₁₀; and no CoQ₉ could be detected in the heart as most of the CoQ₉ was detected as CoQ₁₀. The results thus, raises interesting possibility that nutritionally supplemented CoQ₉ could be an economic alternative to CoQ₁₀ and CoQ₉ could provide enhanced levels of CoQ₁₀ in the mammals and provide cardioprotection after being converted into CoQ₁₀.

The intricate details of the outcome of the experiments corresponding to different aspects of the present invention are described below.

### Effects of CoQ₉/CoQ₁₀ on the Recovery of Left Ventricular Function.

Table 1 shows the recovery of post-ischemic cardiac function in isolated hearts subjected to 30 min ischemia followed by 120 min of reperfusion obtained from guinea pigs treated with 5 mg/kg/day of CoQ₁₀ and CoQ₉, respectively, for 4 weeks. The results clearly show that post-ischemic recovery in heart rate (HR), coronary flow (CF), aortic flow (AF), and left ventricular developed pressure (LVDP) were significantly improved in the CoQ₁₀ and CoQ₉ treated groups in comparison with the drug-free control values. Thus, for instance, after 30 min of ischemia followed by 120 min of reperfusion, aortic flow (Table 1) was significantly increased from its drug-free control value of 8.0±1.0 ml/min to 18.0±2.0 ml/min (*p<0.05) and 26.0±1.0 ml/min (*p<0.05) in hearts obtained from guinea pigs treated with 5 mg kg day of CoQ₁₀ and CoQ₉ respectively. Similar types of post-ischemic recovery of HR, CF, and LVDP were registered (Table 1) in isolated hearts obtained from guinea pigs treated with 5 mg/kg/day of CoQ₁₀ or CoQ₉ for 4 weeks. The improvement in post-ischemic cardiac function (HR, CF, AF, and LVDP) was more pronounced in the CoQ₉ treated group than in the CoQ₁₀ treated group. However, before ischemia, cardiac function (HR, CF, AF, and LVDP) was not significantly changed in the CoQ₁₀ or CoQ₉ treated groups in comparison with the drug-free control values (Table 1).

### Effects of CoQ₉/CoQ₁₀ on the Development of Arrhythmias

The incidence of reperfusion-induced VF was significantly reduced by CoQ₁₀ and CoQ₉. As shown in FIG. 1, and compared to untreated ischemic/reperfused drug-free group, incidence of reperfusion-induced VF was reduced from 92% to 25% (*p<0.05) and 92% to 8% (*p<0.05) with 5 mg/kg/day of CoQ₁₀ and CoQ₉, respectively.

### Effects of CoQ9/CoQ10 on Myocardial Infarct Size.

FIG. 2/A shows the percentage of infarct size in isolated guinea pig hearts subjected to 30 min of global ischemia followed by 120 min of reperfusion. Drug-free ischemic/reperfused control hearts were associated with a 38±4.1% infarct size (FIG. 2/A) which was consistently reduced by the dose of 5 mg/kg/day of CoQ₁₀ and CoQ₉ to 21.1±5% (*p<0.05) and 16.3±3.2% (*p<0.05), respectively.

### Effects of CoQ₉/CoQ₁₀ on Myocardial Apoptosis

As shown in FIG. 2B, in case of ischemic control group guinea pig (I/R) cardiomyocyte apoptosis determined by Tunel method was about 21±2% at the end of reperfusion. Both CoQ₁₀ and CoQ₉ treatment significantly reduced the number of apoptotic cardiomyocytes to 6±1% (p<0.05) and 7±1.5% (p<0.05) respectively.

### LC Analysis of CoQ9/CoQ10.

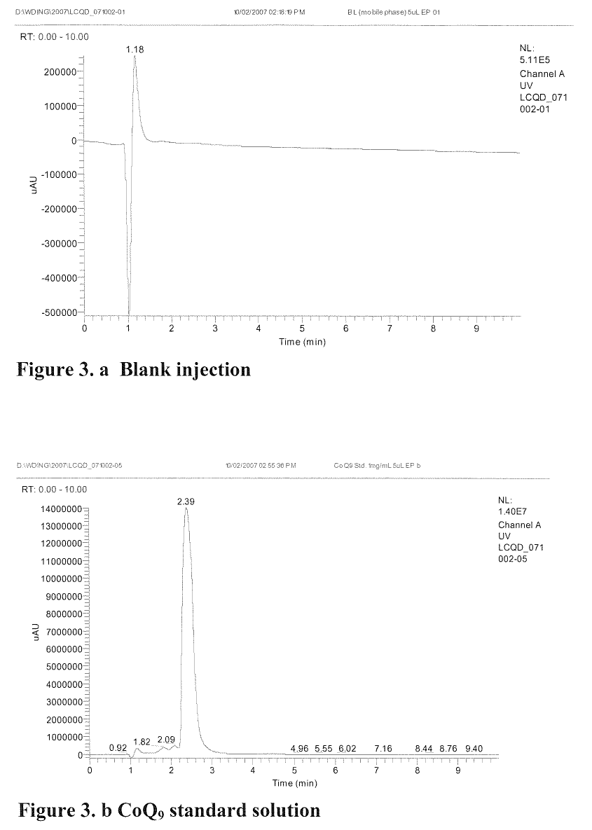

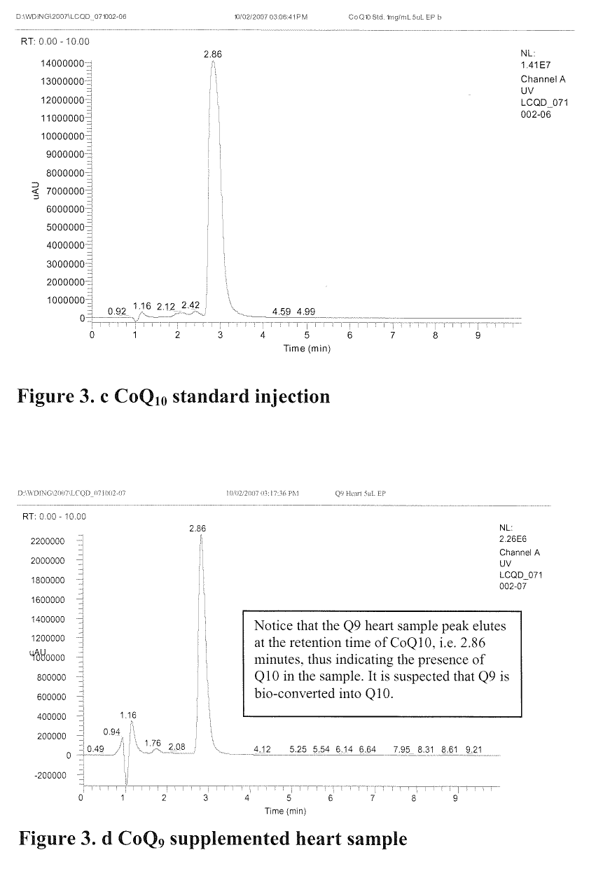

CoQ₉ and CoQ₁₀ were observed at the retention times of 2.39 and 2.86 minutes respectively. FIGS. 3b and 3c shows chromatograms of CoQ₉ and CoQ₁₀ standard solutions. However, the retention time of CoQ₉ heart sample indicated a retention time of 2.86 minutes and not 2.39 minutes (FIG. 3d). The retention time of the CoQ₉ heart sample matched with that of CoQ₁₀ rather than CoQ₉. The qualitative analysis was done by identifying the compounds by their retention times. It was obvious that at this point CoQ₉ was probably bio-converted to CoQ₁₀. Further investigation was conducted by using mass spectrometry to verify the conversion of Q₉ into Q₁₀ in the heart sample.

### Mass Spectroscopy of CoQ9/CoQ10.

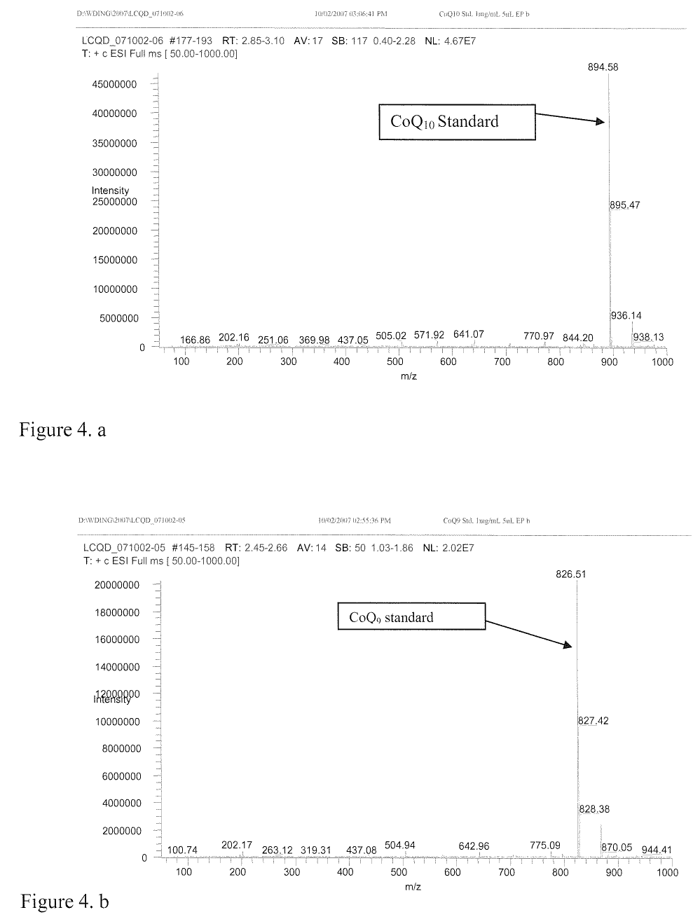

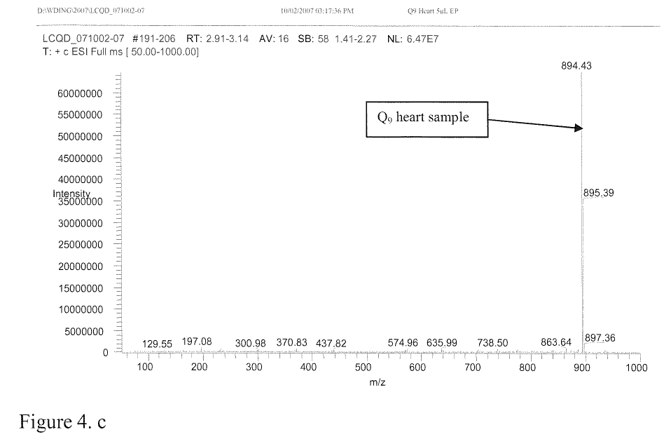

The analytical sensitivity for CoQ₁₀ is known to be very low due to poor ionization property of CoQ₁₀ [Teshima, K., et al. Anal. Biochem. 2005, 338, 12-19]. Hence the optimization of the LC-MS method was done by introducing 5 mmol of methylamine (v/v/v) in the mobile phase, to enhance the sensitivity for-the determination of CoQ₉ and CoQ₁₀. The standard and heart derived sample solutions were injected using an Agilent 1100 HPLC. The HPLC was interfaced with the mass spectrometer and electron spray ionization mass spectrometry (ESI-MS) was conducted for the identification of the compounds. The ion spectra of standard samples of CoQ₉ and CoQ₁₀ exhibited [M+CH₃NH₃]⁺ peaks at m/z 826.5 and m/z 894.6 respectively (FIG. 4b and 4c). However, the CoQ₉ heart sample indicated a mass peak at m/z 894.6 (see FIG. 4), which matches the m/z peak for CoQ₁₀ and not CoQ₉ (FIG. 4d). Therefore, there is evidence that CoQ₁₀ is present in the Q9 supplemented heart sample. This confirms the bio-conversion of CoQ₉ into CoQ₁₀.

CoQ₁₀ is present ubiquitously in most of the mammals including humans except for rodents where CoQ₉ is the predominant form of CoQ. For this reason, the inventors choose guinea pigs as experimental animals to study the effect of CoQ₉ as the hearts of this animal does not contain any CoQ₉. Feeding the guinea pigs CoQ₉ for 4 weeks provided similar degree of cardioprotection as CoQ₁₀. Since most of the CoQ9 was found as CoQ₁₀, it could be possible that CoQ₉ after being converted into CoQ₁₀ provided cardioprotection. In addition the present invention provides valuable information that nutritional supplementation of CoQ₉ should be adequate for the animals needing CoQ₁₀ supplementation.

The generation of CoQ₁₀ is a complex process requiring many cofactors (e.g., vitamin B₆, B₁₂, folic acid, etc.) and several chain reactions. In the present study, prior to subjecting the hearts to ischemia/reperfusion protocol, majority of CoQ₉ was found to be present as CoQ₁₀.

It is generally accepted that most of the exogenously administered CoQ₁₀, either as nutritional supplement or derived from CoQ₁₀ rich foods, is taken up by the liver and blood components, and only a small amount goes to other organs such as heart. In the present study, the inventors were able to detect appreciable amount of CoQ₁₀ in the heart tissue and very small or no amount of CoQ₉ after 4 weeks of CoQ₉ supplementation.

In summary, the results of the present study demonstrate for the first time that nutritional supplementation of CoQ₉ leads to enrichment of CoQ₁₀ levels in the mammal and also that nutritional supplementation of CoQ₉ can reduce myocardial ischemia reperfusion injury to the same extent as CoQ₁₀. The cardioprotection was achieved either directly from CoQ₉ or indirectly through its bioconversion into CoQ₁₀. Nevertheless, the finding that CoQ₉ and CoQ₁₀ can provide the same degree of cardioprotection appears to be important due the fact that only very little exogenous CoQ₁₀ is taken up by the heart, while significant amount of CoQ₁₀ was detected in the heart after four weeks of CoQ₉ feeding. It is tempting to speculate that heart may be able to better utilize CoQ₉ than CoQ₁₀.

To obtain full benefit, it is preferable that the CoQ₉ ingredient is used as it is or can be formulated into a solid, semi-solid or liquid dosage form by adding a conventional biologically acceptable carrier or diluent.

Specific form includes, for example, oral agents such as tablets, soft capsule, hard capsule, pills, granules, powders, emulsions, suspensions, syrups, and pellets; and parenteral agents such as injections, drops, suppositories and the like.

The CoQ₉ ingredient may be optionally combined with suitable quantity of CoQ₁₀ and the composition obtained thereof is administered using a method described above.

The CoQ₉ composition or formulation used in the present invention may be prepared by formulating CoQ₉ along with the biologically acceptable carrier or diluents.

The examples of the biologically acceptable carrier or diluents employed in the present inventions includes but are not limited to, surfactants, excipients, binders, disintegrators, lubricants, preservatives, stabilizers, buffers, suspensions and drug delivery systems.

Preferred examples thereof include solid carriers include glucose, fructose, sucrose, maltose, sorbitol, stevioside, corn syrup, lactose, citric acid, tartaric acid, malic acid, succinic acid, lactic acid, L-ascorbic acid, dl-.alpha.-tocopherol, glycerin, propylene glycol, glycerin fatty ester, polyglycerin fatty ester, sucrose fatty ester, sorbitan fatty ester, propylene glycol fatty ester, acacia, carrageenan, casein, gelatin, pectin, agar, vitamin B group, nicotinamide, calcium pantothenate, amino acids, calcium salts, pigments, flavors, and preservatives. Preferred examples of liquid carriers (diluents) include distilled water, saline, aqueous glucose solution, alcohol (e.g. ethanol), propylene glycol, and polyethylene glycol; and oily carriers such as various animal and vegetable oils, white soft paraffin, paraffin and wax.

In alternative aspects of the invention, the product of the present invention is delivered in the form of controlled release tablets, using controlled release polymer-based coatings by the techniques known in the art. The said formulation is designed for once daily administration.

In other aspects of the invention, the product of the present invention is delivered in the form of nanoencapsulated or liposomal formulation to enhance the solubility and bioavailability.

In accordance to the present invention, the CoQ₉ or the composition is formulated into any food and drink forms such as solid food like chocolate or nutritional bars, semisolid food like cream or jam, or gel. Contemplation was also done to formulate the product of the invention into a beverage and the like, such as refreshing beverage, coffee, tea, milk-contained beverage, lactic acid bacteria beverage, drop, candy, chewing gum, chocolate, gummy candy, yoghurt, ice cream, pudding, soft adzuki-bean jelly, jelly, cookie and the like. These various preparations or foods and drinks are useful as a healthy food for the treatment and prevention of cardiac problems.

The method of enriching CoQ₁₀ teaches that the amount of the CoQ₉ or its composition to be administered or ingested to mammals in the form of above-mentioned formulations or preparations or foods and drinks is not uniform and varies depending on the nature of the formulation and suggested human or animal dosage of CoQ₉, but preferably within a range from 0.01 to 300 mg/kg weight/day.

In a further variation of the invention, the CoQ₉ or the composition containing CoQ₉ used for the supplementation, may optionally combined with a suitable quantity of CoQ₁₀.

The present invention is illustrated by the following non-limiting examples;

### EXAMPLE 1

Protective effect of CoQ9 and CoQ10 against from Ventricular Fibrillation (VF): Healthy Male Hartley guinea pigs of about 350-400 gm body weight were randomly divided into three groups, Control, CoQ₉ and CoQ₁₀. The guinea pigs were given orally 5 mg/kg body weight [in 0.5 ml water] of vehicle only, CoQ₉ or CoQ₁₀ respectively by gavage once a day. CoQ₉ or CoQ₁₀ by gavaging once a day 5 mg/kg [in 0.5 ml water] body weight. Treatment was continued for 30 days, the animals had free access to food and water. After 30 days, all animals were anesthetized, heparinized and sacrificed. The hearts excised, and isolated for perfusion via Langendorff mode for 5 min of washout period of the Langendorff heart perfusion, the pulmonary vein was cannulated, and the heart was switched to the “working” mode via perfusion of the left atria (at a filling pressure of 17 cm of the buffer, 1.7 kPa) as it was described in detail elsewhere. Global ischemia was imposed by clamping the atrial and aortic cannulas. Epicardial ECG was recorded through out the experiment, by attaching two silver electrodes directly to the myocardium and data collected using a data acquisition system (ADInstruments, Powerlab, Castle Hill, Australia). ECGs were analyzed to determine ventricular fibrillation (VF) and ventricular tachycardia (VT). The first 10 min of reperfusion was done in Langendorf (‘nonworking’) mode in order to avoid the development of reperfusion-induced VT and VF during the ‘working’ heart reperfusion. After the initial 2 min of VT or/and VF (sustained VF) in Langendorff reperfusion, hearts were defibrillated (if it was necessary), reperfused for an additional 8 min in Langendorff mode, and switched to ‘working heart’ reperfusion, and myocardial function was recorded. The heart was considered to be in VF if an irregular undulating baseline was apparent on the ECG. The data of VT, VF and sinus rhythm show their durations (in seconds) within the first 120 s of nonworking Langendorff reperfusion. The incidences of reperfusion-induced ventricular fibrillation (VF) for CoQ9 and CoQ10 are depicted in FIG. 1. Pretreatment of CoQ9 and CoQ10 significantly reduced the incidence of ischemia-reperfusion induced ventricular fibrillation (VF), compared to untreated drug flee group. Incidence of VF was reduced from 92% (control group) to 25% (*p<0.05) and 8% (*p<0.05) with 5 mg/kg/day of CoQ₁₀ and CoQ₉, respectively.

### EXAMPLE 2

CoQ9 and CoQ10 treatment protects from cardiac infarction: Animal preparation, drug pretreatment and isolated working heart preparation were done as described in example 1. Animal pretreatment and isolated heart experiments were done as mentioned in example 1. Hearts for determination of infarct size were perfused, at the end of each experiment, with 25 ml of 1% tniphenyl tetrazolium solution (TTC) in phosphate buffer (Na₂HPO₄ 88 mM, NaH₂PO₄ 1.8 mM) via the side arm of the aortic cannula, and then stored at −70° C. for later analysis. Frozen hearts were sliced transversely in a plane perpendicular to the apico-basal axis into 3-4 mm thick sections, weighted, blotted dry, placed in between microscope slides and scanned on a Hewlett-Packard Scanjet 5p single pass flat bed scanner (Hewlett-Packard, Palo Alto, Calif., USA). Using the NIH Image 1.61 image processing software, Infarct zones of each slice were traced and the respective areas were calculated in terms of pixels. The areas were measured by computerized planimetry software and these areas were multiplied by the weight of each slice, then the results summed up to obtain the weight of the risk zone. Infarct size was calculated as the ratio, in percent, of the infarct zone to the risk zone. Effects of CoQ₁₀ and CoQ₉ on infarct size in isolated guinea pig hearts are depicted in FIG. 2A.

Pretreatment of CoQ9 and CoQ10 significantly reduced global ischemia induced cardiac infarction compared to untreated drug free group. Drug-free ischemic/reperfused control hearts were associated with a 38±4.1% infarct size which was consistently reduced by the dose of 5 mg/kg/day of CoQ₁₀ and CoQ₉ to 21.1±5% (*p<0.05) and 16.3±3.2% (*p<0.05), respectively.

### EXAMPLE 3

CoQ9 and CoQ10 treatment reduces apoptosis of cardiomyocytes: Animal preparation, drug pretreatment and isolated working heart preparation were done as described in example 1. Immunohistochemical detection of apoptotic cells was carried out using TUNEL assay, using APOPTAG® kit (Oncor, Gaithersburg, Md.). The heart tissues were immediately put in 10% formalin and fixed in an automatic tissue-fixing machine. The tissues were embedded in the molten paraffin in metallic blocks. Prior to analyzing tissues for apoptosis, tissue sections were deparaffinized with xylene and washed in succession with different concentrations of ethanol (absolute, 95%, 70%). Then tissues were incubated with mouse monoclonal antibody recognizing cardiac myosin heavy chain to specifically recognize apoptotic cardiomyocytes. The fluorescence staining was viewed with a confocal laser microscope. The number of apoptotic cells was counted and expressed as a percent of total myocyte population. Effects of CoQ₁₀ and CoQ₉ on cardiomyocyte apoptosis in isolated guinea pig hearts are depicted in FIG. 2B Pretreatment with CoQ9 and CoQ10 significantly reduced the incidence of ischemia-reperfusion induced apoptosis of cardiomyocytes compared to untreated drug free group. Apoptosis of cardiomyocytes determined by Tunel method in control group was about 21±2% at the end of reperfusion. Both CoQ₁₀ and CoQ₉ treatment significantly reduced the number of apoptotic cardiomyocytes to 6±1% and 7±1.5% respectively.

### EXAMPLE 4

CoQ9 and CoQ10 treatment improves post-ischemic cardiac function (HR, CF, AF, and LVDP): Animal preparation, drug pretreatment and isolated working heart preparation were done as described in example 1. The isolated hearts obtained from group II and Group III guinea pigs treated with 5 mg/kg/day of CoQ₁₀ and CoQ₉, respectively, for 4 weeks were subjected to 30 min ischemia followed by 120 min of reperfusion. The recovery of post-ischemic cardiac function in isolated hearts was evaluated by measuring various parameters including Coronary Flow (CF), Aortic Flow (AF), Left Ventricular Developed Pressure (LVDP), and Heart rate (HR) before ischemia, after 60 min of reperfusion and after 120 min of reperfusion using Langendroff apparatus. The results are summarized in table 1. The pretreatment with CoQ9 and CoQ10 significantly improved the post-ischemic recovery in HR, CF, AF, and LVDP compared to the drug-free control group. Pretreatment with CoQ9 and CoQ10 significantly protected the heart from decrease in all functional parameters induced by ischemia-reperfusion. The improvement in post-ischemic cardiac function (HR, CF, AF, and LVDP) was more pronounced in the CoQ₉ treated group than it was registered in the CoQ₁₀ treated group.
a. Coronary flow: The reduction in coronary flow due to ischemia-reperfusion was significantly protected in CoQ10 (19±1) and CoQ 9 (25±2) treated groups in comparison to drug free control group (15±1). However CoQ9 completely improved CF to its normal value recorded before ischemic reperfusion (Before ISA 23±2 After RE 25±2) where as CoQ 10 did not improved CF to normal state (Before ISA 25±2 After RE 19±1).
b. Aortic flow: The reduction in aortic flow due to ischemia-reperfusion was significantly protected in CoQ10 (18±2) and CoQ 9 (26±1) treated groups in comparison to drug free control group (8±1).
c. Left ventricular developed pressure: The reduction in LVDP due to ischemia-reperfusion was significantly protected in CoQ10 (64±3) and CoQ 9 (75±2) treated groups in comparison to drug free control group (45±3).
d. Heart rate: The reduction in heart rate due to ischemia-reperfusion was significantly protected in CoQ10 (217±3) and CoQ9 (233±4) treated groups in comparison to drug free control group (182±4).

### Statistics:

The values of HR, CF, AF, LVDP, and infarct size were expressed as mean value±SEM. A two-way analysis of variance was first carried out to test for any differences in mean values between groups. If differences were established, the values of the drug-treated groups were compared with those of the drug-free group by Dunnett's test. A different procedure, because of the nonparametric distribution, was used for the distribution of discrete variables, such as the incidence of VF. Thus, the chi-square test was used to compare the incidence of VF between untreated-control and treated groups.

### EXAMPLE 5

### High Performance Liquid Chromatography [HPLC] and Mass Spectroscopy [MS] for the Determination of CoQ₉ and CoQ₁₀:

Preparation of CoQ₉ and CoQ₁₀ heart samples: Animal preparation, drug pretreatment and isolated working heart preparation were done as described in example 1. The study animals at the end of four week period were anesthetized by heparin administration and then the animals were scarified, and the hearts excised. The ground heart samples provided for analysis were centrifuged at 3000 rpm for 10 minutes. The supernatant was then transferred to another centrifuge tube and was evaporated to dryness using nitrogen, in order to obtain a more concentrated solution. The residue after dryness was then dissolved using 2 ml of mobile phase, and was then transferred to an autosampler injection vial. The samples were analyzed immediately after preparation, and the remainder of the standard solutions was stored at 5° C. for future analysis.

### Preparation of CoQ9 and CoQ10 Standard Solutions:

Standard solutions were prepared by weighing approximately 10 mg of CoQ₉ and CoQ₁₀ standards respectively into a 100 ml volumetric flask and then dissolving it by using the mobile phase as a diluent. The stock solution was further diluted 1:10 to attain a final working concentration of 0.01 mg/ml. The CoQ₁₀ stock solution was sonicated for 5 minutes for complete dissolution of the powder into solution.

### HPLC Analysis of CoQ₉ and CoQ₁₀:

The modular HPLC system consisted of an Agilent 1100 quaternary pump, Agilent 1100 autosampler, Agilent 1100 column heater, and Agilent 1100 UV detector. The analysis of CoQ₉ and CoQ₁₀ was performed by using a YMC Pro C18, 3 μm, 120° A, 2.0×50 mm column and the mobile phase consisted of methanol-(2-propanol)-formic acid (45:55:0.05, v/v/v), containing methylamine at the concentration of 5 mmol/L. The flow rate was 0.2 ml/min and the column compartment was maintained at 40° C. The injection volume was 5 μl [13].

The HPLC chromatograms for the standard sample of CoQ₉ and CoQ₁₀ are depicted in FIGS. 3b and 3c respectively. CoQ₉ and CoQ₁₀ were observed at the retention times of 2.39 and 2.86 minutes respectively. The samples obtained from the ground hearts of control group (group I), CoQ₉ supplemented group (group II) and CoQ₁₀ supplemented group (group III) were analyzed and the control chromatogram was subtracted from that of the CoQ₉ fed sample and CoQ₁₀ fed sample and the HPLC chromatograms for the heart samples of CoQ₉ and CoQ₁₀ are depicted in FIGS. 3d and 3e respectively. The HPLC chromatogram for the heart sample from the animals supplemented with CoQ₉ showed significantly high enrichment in the CoQ₁₀ content over and above the natural concentration as indicated by an intense peak at 2.86 (FIG. 3d). Its identity was further conformed by mass spectrometric analysis. It showed a mass peak at m/z 894.6 for CoQ₁₀ [M+CH₃NH₃]⁺ and it matches with that observed for a standard sample of CoQ₁₀.

### EXAMPLE 6

Mass Spectroscopy for the Identification of the Peaks: Finnigan LCQ ion trap bench top mass spectrometer (Thermo Fischer Scientific, Mass., USA) interfaced with an Agilent 1100 HPLC system was used for analysis. Data processing was done in the Finnigan Xcalibur data system operating on Windows® NT PC-based system. The turbo ion spray interface and mass spectrometer were operated under the following conditions: positive ionization polarity, 4.8 kV spray voltage, 425° C. probe temperature, collision gas pressure, 2.8×10⁻⁵ Torr [13]. All parameters were adjusted for each analyte, using the tune method CoQ₁₀ EP071002 created by the analyst at the time of analysis with the Xcalibur software. Divert valve and contact closure were not used during the run. Optimization of the LC-MS method was done by introducing 5 mmol of methylamine (v/v/v) in the mobile phase, to enhance the sensitivity for the determination of CoQ₉ and CoQ₁₀. The standard and sample solutions were injected using an Agilent 1100 HPLC. The flow rate of 0.2 ml/min. was maintained. A YMC Pro C18, 3 μm, 120° A, 2.0×50 mm column was used. The HPLC was interfaced with the mass spectrometer. Electron spray ionization mass spectrometry (ESI-MS) was conducted for the identification of the compounds. A full MS scan from 50 to 1000 units was run to obtain the m/z ratios for the compounds of interest, namely CoQ₉ and CoQ₁₀. No MS/MS or fragmentation was done at this point. In the presence of methylamine in the mobile phase, the product ion spectra of both [M+CH₃NH₃]⁺ at m/z 826.5 for CoQ₉ and m/z 894.6 for CoQ₁₀ was observed (see FIGS. 4a and 4b). However, the CoQ₉ heart sample indicated a mass peak at m/z 894.6 (see FIG. 4c), which matches the m/z peak for CoQ₁₀ and not CoQ₉.

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

TABLE 1
Cardiac function in isolated ischemic/reperfused hearts obtained from guinea pigs
treated with 5 mg/kg/day of Q10 and Q09, respectively, for 4 weeks.

| | Before ISA | | | | After 60 min of RE | | | | After 120 min of RE | | | |
|---|---|---|---|---|---|---|---|---|---|---|---|---|
| Group | HR | CF | AF | LVDP | HR | CF | AF | LVDP | HR | CF | AF | LVDP |
| Control | 254 ± 5 | 24 ± 1 | 32 ± 2 | 104 ± 5 | 195 ± 4 | 15 ± 1 | 7 ± 1 | 49 ± 3 | 182 ± 4 | 15 ± 1 | 8 ± 1 | 45 ± 3 |
| 5 mg/kg | 261 ± 6 | 25 ± 2 | 34 ± 2 | 100 ± 5 | 228 ± 3* | 20 ± 1* | 20 ± 2* | 71 ± 3* | 217 ± 3* | 19 ± 1* | 18 ± 2* | 64 ± 3* |
| Q10 | | | | | | | | | | | | |
| 5 mg/kg | 248 ± 5 | 23 ± 2 | 35 ± 2 | 106 ± 4 | 240 ± 4* | 22 ± 2* | 27 ± 1* | 80 ± 2* | 233 ± 4* | 25 ± 2* | 26 ± 1* | 75 ± 2* |
| Q9 | | | | | | | | | | | | |

n = 12 in each group;
heart rate (HR) beats/min;
coronary flow (CF) ml/min;
arotic flow (AF) ml/min;
Lleft ventricular developed pressure (LVDP) mm Hg;
ischemia (ISA);
reperfusion (RE).
*p < 0.05 compared to the values of the control group

1. A method of increasing CoQ10 concentration in mammals, comprising administering to a mammal a therapeutically effective amount of CoQ9.

2. The method as claimed in claim 1, wherein the therapeutically effective amount of the CoQ9 ranges from about 0.01 to about 50 mg/kg/day.

3. The method as claimed in claim 1, wherein the therapeutically effective amount of the CoQ9 is about 5 mg/kg/day.

4. A method as claimed in claim 1, wherein the CoQ9 is administered in the form of a pharmaceutical composition further comprising a pharmaceutically acceptable carrier or diluent and optionally further comprising one or more additives selected from the group consisting of surfactants, excipients, binders, disintegrants, lubricants, preservatives, stabilizers, and buffers.

5. The method as claimed in claim 4, wherein the CoQ9 is orally administered.

6. The method as claimed in claim 5, wherein the CoQ9 is orally administered as a dosage form selected from the group consisting of tablets, soft capsules, hard capsules, pills, granules, powders, emulsions, suspensions and pellets.

7. The method as claimed in claim 4, wherein the CoQ9 is parenterally administered.

8. The method as claimed in claim 7, wherein the CoQ9 is parenterally administered as a dosage form selected from the group consisting of injections, drops, and suppositories.

9. The method as claimed in claim 4, wherein the CoQ9 is administered in the form of a drug-delivery system.

10. The method as claimed in claim 9, wherein the drug-delivery system is selected from the group consisting of microencapsulated drug-delivery systems, nanoparticle-based drug-delivery systems, liposome-based drug-delivery systems, biodegradable block copolymer drug-delivery systems, and polymeric surfactant-based drug-delivery systems.

11. The method as claimed in claim 1, wherein the CoQ9 is orally administered in the form of a dietary supplement composition, a food composition, or a nutraceutical composition.

12. The method as claimed in 11, wherein the CoQ9 is orally administered in the form of a composition selected from the group consisting of nutritional bars, creams, jams, gels, candies, chewing gums, cookies, and beverages.

13. The method as claimed in claim 1, wherein the CoQ9 is topically administered.

14. The method as claimed in claim 13, wherein the CoQ9 is topically administered in the form of a cosmetic composition.

15. The method as claimed in claim 1, comprising administering the CoQ9 in combination with CoQ10.

16. The method as claimed in claim 15, comprising administering from about 0.01 to about 50 mg/kg/day of the CoQ9 and from about 0.01 to about 50 mg/kg/day of the CoQ10.

17. The method as claimed in claim 15, comprising administering about 5 mg/kg/day of the CoQ9 and about 5 mg/kg/day of the CoQ10.
